(19) United States
(12) Patent Application Publication
(10) Pub. No.: US 2009/0092623 A1
(43) Pub. Date: Apr. 9, 2009

(54) Promoter

(76) Inventors: Gerald Wayne Gough, Hertfordshire (GB); Christopher Michael Roberts, Hertfordshire, (GB)

Correspondence Address: GLAXOSMITHKLINE;Corporate Intellectual Propewrty UW2220, P.O. Box 1539, Kindgof Prussia,, PA, 19406-0939, US

(21) Appl. No.: 10/586,380

(22) PCT Filed: Jan. 13, 2005
(86) PCT No.: PCT/EP05/00443

§ 371 Date: Aug. 18, 2008

(30) Foreign Application Priority Data
Jan. 16, 2004 (GB) 0400965.0

(51) Int. Cl. A61K 39/00; C12N 15/00; C12N 5/06; A61M 37/00; C12P 21/04; A61K 31/7088

(52) U.S. Cl. 424/184.1; 435/320.1; 514/44; 435/325; 435/69.1; 604/304

(57) ABSTRACT

The present invention provides a novel polynucleotide vectors and their use in the production of biological material in host cells, and also in medical therapy or polynucleotide vaccination. The novel vectors of the present invention comprise a promoter normally associated with the US3 gene of Human Cytomegalovirus (HCMV).

The present invention provides a novel polynucleotide vectors and their use in the production of biological material in host cells, and also in medical therapy or polynucleotide vaccination. The novel vectors of the present invention comprise a promoter normally associated with the US3 gene of Human Cytomegalovirus (HCMV). Preferably the vectors comprising the HCMV US3 gene promoter are plasmids or viral or bacterial vectors that comprise a polynucleotide sequence that encode at least one polypeptide which is not an HCMV US3 protein, which viral or bacterial vector or plasmid are used for vaccination purposes.

The major immediate-early gene promoter of human cytomegalovirus (HCMV MIE) has been extensively characterised. In addition, the HCMV genome also has at least three other immediate-early promoters. Among these the HCMV US3 gene has a large and complex promoter region (Weston. K 1988, Virology 162: 406-416). The expression of the US3 gene is controlled by its promoter comprises a minimal promoter region, an enhancer region (R2) and, silencer region (R1).

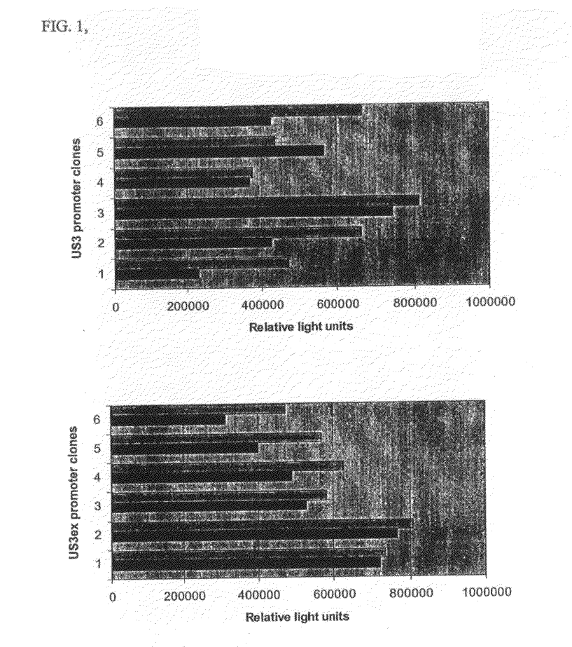

The US3 promoter comprises about 700 bp cis-acting regulatory domain, about 600 bp upstream of the transcription start site, and about 100 bp downstream of said site. The R2 region contains multicopy NF-kappa B binding sites known to confer high basal expression in transfected human cell lines. The R1 region contains multiple repeats of a 10-bp TGTCGCGACA palindromic motif that also contains a Nru restriction enzyme site. The R1 silencer element has been shown to down regulate heterologous promoters as well as the minimal US3 promoter element in transient transfection experiments (Chan Y-J et al 1996. J. Virol 70: 5312-5328). Within the context of the viral genome however the R1 element appears to increase expression of a reporter (CAT) gene (Bullock G C 2001. Virology 288: 164-174). One possible function for the R1 element may be the maintenance of a chromatin free region so facilitating transcriptional activation of the adjacent R2 enhancer (Bullock G C et al 2002. Exp Mol Pathol 72: 196-206). Within the US3 promoter of the Towne strain of HCMV, the R1 silencer region is located at position −314 to −596 and, the R2 enhancer region resides from −313 to −55, the minimal promoter is from −54 to +80 (Chan Y-J et al 1996. J. Virol 70: 5312′-5328). An EcoRV restriction site lies at position −316 to −311 and operationally defines the boundary between the Nm (R1) and NF-kappa B (R2) regions. All map positions are relative to the transcription start site at +1 (see FIG. 1B of Chan et al. supra). The US3 promoters derived from other strains of HCMV (e.g. AD169 and Toledo) follow the same overall architecture, however, the numbering of the base positions will be slightly different.

A cis repression sequence (CRS), has been described in the US3 promoter (CGTGCAGTCCACACG) located immediately upstream of the transcription start site and, a consensus initiator-like (Inr) element (CTACTTC) immediately downstream of the transcription start site. The viral IE86 protein binds to the CRS element and contributes to promoter repression early after viral infection of permissive cells (Lashmit P E et al, 1998. J. Virol 72, 9575-9584). An additional cis repression element between the transcription start site and TATA box is bound by the sequence-specific viral DNA-binding protein UL34 and can represses transcription of the US3 gene product perhaps by preventing formation of the transcriptional initiation complex (Lapierre, L. A., et al., 2001, J. Virol., 75, 6062-6069).

The US3 gene is transcribed with immediate-early kinetics with first appearance of transcripts at 1 hour-post infection and maximal expression occurs between 2 and 5 hours post infection in permissive cells with a decline thereafter (Tenney D J et al 1991. J. Virol 65: 6724-6734). Three alternatively spliced transcripts are generated and likely encode related but distinct proteins. The full length transcript is the most abundant, encoding a 22 kd protein which specifically retains class 1 molecules in the ER (Wenzhong L et al 2002. Virology 301: 32-42).

The US3 gene is known to cause retention of MHC class 1 heavy chains in the ER so preventing the presentation of viral antigens on the surface of infected cells (Ahn K A et al 1996. PNAS 93: 10990-10995). The US3 gene is one of several immune evasion genes encoded by HCMV.

In one embodiment of the present invention there is provided novel vectors which cause the expression of polypeptides in a host cell, wherein the vectors include the promoter element of the Human Cytomegalovirus (HCMV) US3 gene, the promoter being operably linked to a region encoding a heterologous polypeptide which is foreign with respect to the HCMV US3 protein.

In another embodiment of the present invention there is provided novel vectors which cause the expression of polypeptides in a host cell, wherein the vectors include a promoter comprising the minimal promoter element of the Human Cytomegalovirus (HCMV) US3 gene and a transcription regulatory element, the US3 minimal promoter element being operably linked to a region encoding a heterologous polypeptide which is foreign with respect to the HCMV US3 protein.

Enhancers are cis-acting elements of DNA that stimulate transcription of adjacent genes by RNA polymerase II, in either orientation, and over distances up to several kilobase pairs, even from a position downstream of the transcribed region (Boshart et al., 1985, Cell, 41, 521-530).

In one embodiment the polynucleotide vector comprises the minimal promoter element of the HCMV US3 gene and a transcription regulatory element which is an enhancer element. In this embodiment the novel vectors of the present invention containing the minimal HCMV US3 promoter may further comprise the R2 enhancer element of the HCMV US3 gene. For example, the HCMV US3 R2 enhancer element will be positioned immediately upstream of the minimal HCMV US3 minimal promoter. Alternatively, the vectors of the present invention may comprise the minimal promoter element of the HCMV US3 gene together with a transcription regulatory element normally associated with another gene, for example the HCMV major immediate-early protein gene enhancer (see U.S. Pat. No. 5,168,062 and U.S. Pat. No. 6,218,140).

In one aspect of the present invention, the vectors and US3 promoters are provided wherein the US3 promoter does not comprise the R1 silencer element. Alternatively the US3 promoter is mutated such that the silencing effect of R1 is reduced or abrogated for example the US3 promoter may be mutated to reduce the number of TGTCGCGACA palindromic motifs that also contains a Nru restriction enzyme site, in one embodiment all of said motifs are removed (Chan et al., supra). In an alternate embodiment, the promoter can comprise the minimal promoter element and R2 elements from HCMV US3, together with a active or disabled silencer element from a non-HCMV US3 gene promoter.

The sequences of the US3 minimal promoter and R2 enhancer elements are provided herein and in the examples, and in one embodiment are derived from the Toledo strain of HCMV. It is intended that sequences derived from other strains of HCMV also form part of the present invention.

In an alternative embodiment of the present invention the vectors comprise the R2 enhancer region of US3 promoter as the only HCMV US3 sequence, together with a minimal promoter derived from a non-HCMV US3 promoter. For example, the vectors of this aspect of the present invention have a promoter comprising HCMV US3 R2 region and the minimal promoter element from HCMV MIE gene promoter.

The HCMV US3 promoter for use in the vectors of the present invention may be derived from the region of between approximately or between positions −600 to +100, relative to the transcription start site of the US3 gene. In another embodiment a smaller US3 promoter is selected from within this region which is downstream of the BcoRV restriction site (for example, for the Towne strain of HCMV, this corresponds to the region between positions −313 to +80, relative to the transcription start site). In another embodiment the vectors contain a promoter that comprises the US3 minimal promoter element as the only sequence that is derived from the HCMV US3 gene promoter, optionally together with an enhancer element from another non-HCMV US3 gene promoter (such as HCMV MIE enhancer). In another embodiment of the present invention, the US3 promoter can comprise a minimal promoter element that is truncated downstream of the transcription start site. In one embodiment said truncated minimal promoter retains the transcription initiation site, which in the Towne strain of HCMV means that the truncation is downstream of the +8 position relative to the transcription start site.

The heterologous protein encoded and expressed by the vectors of the invention are foreign with respect to the HCMV US3 gene product. In one embodiment, “foreign” is intended to mean that the heterologous protein is not an HCMV US3 gene product or a protein having greater than 70% identity to an HCMV US3 gene product.

The novel polynucleotide vectors of the present invention are useful in gene therapy where the vector drives production of a therapeutic protein in a cell; or as polynucleotide vaccines where the plasmid is a polynucleotide immunogen that encodes an antigen, against which it is desired to raise an immune response. The expression vectors of the present invention may also be used for the in vitro expression of therapeutically effective polypeptides.

Within the context of the expression vectors of the present invention, the skilled man is generally aware of those additional elements that are required to create a fully functional expression cassette. For example, it is optimal if the vectors comprise a pol II terminator to terminate transcription and a poly-adenylation signal for stabilization and processing of the 3′ end of an mRNA transcribed from the promoter. Suitable polyadenylation signals include mammalian polyadenylation signals such as, for example, the rabbit beta globin polyadenylation signal or the bovine growth hormone polyadenylation signal and also polyadenylation signals of viral origin, such as the SV40 late poly(A) region. Additionally, the vector preferably comprises a Kozak consensus sequence at the site of initiation of translation.

The US3 gene promoter is expressed in a wide variety of cell types and its early expression kinetics make it an interesting alternative to the HCMV MIE promoter for use in DNA vaccine studies.

In an embodiment, the vector may be a plasmid, a bacterial or viral vector, and in one embodiment is an adeno virus vector or an adeno associated virus vector (AAV).

A plasmid vector may further contain an origin of replication to allow autonomous replication within a prokaryotic host cell and a selective marker, such as an antibiotic resistance gene. Advantageously, one or more restriction sites may be included between the HCMV US3 5′ UTR sequence and the poly-adenylation signal to facilitate insertion of a heterologous coding sequence. Plasmid vectors according to the invention may be easily constructed from the component sequence elements using standard recombinant techniques well known in the art and described, for example, in F. M. Ausubel et al. (eds.), Current Protocols in Molecular Biology, John Wiley & Sons, Inc. (1994).

In another embodiment, the vector may be an expression vector for use in the expression of a recombinant polypeptide in a eukaryotic host cell. In this embodiment the vector may further comprise a DNA sequence encoding a recombinant polypeptide operably linked to the HCMV US3 minimal promoter and 5′ UTR sequence.

The vectors of the present invention may further comprise additional regulatory elements, or sequences, such as the HCMV MIE exon 1 gene sequence, optimally being fused immediately after the transcription initiation sequence (ACGCTACTTCT) of the US3 promoter. The vector may further contain a selective marker which allows selection in eukaryotic host cells, for example a neomycin phosphotransferase marker. The expression vector may also contain one or more further expression cassettes to allow for expression of multiple recombinant polypeptides from a single vector. Most preferably, the expression vector will be a plasmid expression vector.

The DNA sequence encoding the recombinant polypeptide may be essentially any protein-encoding DNA sequence bounded by start and stop codons. This protein-encoding DNA sequence may include introns. In a particularly preferred embodiment the recombinant polypeptide may be an antigenic polypeptide or therapeutic protein.

The term “operably linked” refers to an arrangement in which the polypeptide-encoding DNA sequence is positioned downstream of the promoter and 5′ UTR such that transcription initiation at the transcription start site associated with the promoter results in transcription of an mRNA incorporating the HCMV US3 5′UTR fragment (including any heterologous intron) and the sequence encoding the recombinant polypeptide.

In an embodiment the vectors of the present invention are plasmids that are used as DNA vaccine immunogens. In this context the plasmid encodes a heterologous protein, the expression of which is driven by the US3 promoter as described above, against which it is desired to raise an immune response. The following is a list of pathogens that may be targeted by the vaccines of the present invention, and also a list of potential individual antigens derived from those pathogens that could be encoded by the vectors of the present invention.

In a preferred embodiment the antigen is capable of eliciting an immune response against a human pathogen, which antigen or antigenic composition is derived from HIV-1, (such as tat, nef, gp120 or gp160, gp140, p24, gag, env, vif, polypr, vpu, rev), in this context it is particularly preferred that the HIV antigens, are selected from RT (R), Nef (N) and Gag (G); most preferably the HIV antigen is a fusion protein of all three and is expressed as a single polyproprotein (RNG). Other pathogens include human herpes viruses, such as gH, gL gM gB gC gK gE or gD or derivatives thereof or Immediate Early protein such as ICP27, ICP 47, IC P 4, ICP36 from HSV1 or HSV2, cytomegalovirus, especially Human, (such as gB or derivatives thereof), Epstein Barr virus (such as gp350 or derivatives thereof), Varicella Zoster Virus (such as gpI, II, m and IE63), or from a hepatitis virus such as hepatitis B virus (for example Hepatitis B Surface antigen or Hepatitis core antigen or pol), hepatitis C virus antigen and hepatitis E virus antigen, or from other viral pathogens, such as paramyxoviruses: Respiratory Syncytial virus (such as F and G proteins or derivatives thereof), or antigens from parainfluenza virus, measles virus, mumps virus, human papilloma viruses (for example HPV6, 11, 16, 18, e.g. L1, L2, E1, E2, E3, E4, E5, E6, E7) where it is preferred that the antigens encoded are E1 and E2, flaviviruses (e.g. Yellow Fever Virus, Dengue Virus, Tick-borne encephalitis virus, Japanese Encephalitis Virus) or Influenza virus cells, such as HA, NP, NA, or M proteins, or combinations thereof), or antigens derived from bacterial pathogens such as Neisseria spp, including N. gonorrhea and N. meningitidis, e.g., transferrin-binding proteins, lactoferrin binding proteins, PilC, adhesins); S. pyogenes (for example M proteins or fragments thereof, C5A protease, S. agalactiae, S. mutans; H. ducreyi; Moraxella spp, including M. catarrhalis, also known as Branhamella catarrhalis (for example high and low molecular weight adhesins and invasins); Bordetella spp, including B. pertussis (for example pertactin, pertussis toxin or derivatives thereof, filamenteous hemagglutinin, adenylate cyclase, fimbriae), B. parapertussis and B. bronchiseptica; Mycobacterium spp., including M. tuberculosis (for example ESAT6, Antigen 85A, -B or -C, MPT 44, MPT59, MPT45, HSP10, HSP65, HSP70, HSP 75, HSP90, PPD 19 kDa [Rv3763], PPD 38 kDa [Rv0934]), M. bovis, M leprae, M. avium, M. paratuberculosis, M smegmatis; Legionella spp, including L. pneumophila; Escherichia spp, including enterotoxic E. coli (for example colonization factors, heat-labile toxin or derivatives thereof, heat-stable toxin or derivatives thereof), enterohemorragic E. coli, enteropathogenic E. coli (for example shiga toxin-like toxin or derivatives thereof); Vibrio spp, including V. cholera (for example cholera toxin or derivatives thereof); Shigella spp, including S. sonnei, S. dysenteriae, S. flexnerii; Yersinia spp, including Y. enterocolitica (for example a Yop protein), Y. pestis, Y. pseudotuberculosis; Campylobacter spp, including C. jejuni (for example toxins, adhesins and invasins) and C. coli; Salmonella spp, including S. typhi S. paratyphi, S. choleraesuis, S. enteritidis; Listeria spp., including L. monocytogenes; Helicobacter spp, including H. pylori (for example urease, catalase, vacuolating toxin); Pseudomonas spp, including P. aeruginosa; Staphylococcus spp., including S. aureus, S. epidermidis; Enterococcus spp., including E. faecalis, E. faecium; Clostridium spp., including C. tetani (for example tetanus toxin and derivative thereof), C. botulinum (for example botulinum toxin and derivative thereof), C. difficile (for example clostridium toxins A or B and derivatives thereof); Bacillus spp., including B. anthracis (for example botulinum toxin and derivatives thereof); Corynebacterium spp., including C. diphtheriae (for example diphtheria toxin and derivatives thereof); Borrelia spp., including B. burgdorferi (for example OspA, OspC, DbpA, DbpB), B. garinii (for example OspA, OspC, DbpA, DbpB), B. afzelii (for example OspA, OspC, DbpA, DbpB), B. andersonii (for example OspA, OspC, DbpA, DbpB), B. hermsii; Ehrlichia spp., including E. equi and the agent of the Human Granulocytic Ehrlichiosis; Rickettsia spp, including R. rickettsii; Chlamydia spp., including C. trachomatis (for example MOMP, heparin-binding proteins), C. pneumoniae (for example MOMP, heparin-binding proteins), C. psittaci; Leptospira spp., including L. interrogans; Treponema spp., including T. pallidum (for example the rare outer membrane proteins), T. denticola, T. hyodysenteriae; or derived from parasites such as Plasmodium spp., includingP. falciparum; Toxoplasma spp., including T. gondii (for example SAG2, SAG3, Tg34); Entamoeba spp., including E. histolytica; Babesia spp., including B. microti; Trypanosoma spp., including T. cruzi; Giardia spp., including G. lamblia; Leshmania spp., including L. major; Pneumocystis spp., including P. carinii; Trichomonas spp., including T. vaginalis; Schisostoma spp., including S. mansoni or derived from yeast such as Candida spp., including C. albicans; Cryptococcus spp., including C., neoformans.

Other preferred specific antigens for M. tuberculosis are for example Rv2557, Rv2558, RPFs: RvO837c, Rv1884c, Rv2389c, Rv2450, Rv1009, aceA (Rv0467), PstS1, (Rv0932), SodA (Rv3846), Rv2031c 16 kDal., Th Ra12, Th H9, Th Ra35, Tb38-1, Erd 14, DPV, MTI, MSL, mTTC2 and hTCC1 (WO 99/51748). Proteins for M. tuberculosis also include fusion proteins and variants thereof where at least two, preferably three polypeptides of M. tuberculosis are fused into a larger protein. Preferred fusions include Ra12-TbH9-Ra35, Erd14-DPV-MTI, DPV-MTI-MSL, Erd14-DPV-MTI-MSL-mTCC2, Erd14-DPV-MTI-MSL, DPV-MTI-MSL-mTCC2, TbH9-DPV-MTI (WO 99/51748).

Most preferred antigens for Chlamydia include for example the High Molecular Weight Protein (HWMP) (WO 99/17741), ORF3 (EP 366 412), and putative membrane proteins (Pmps). Other Chlamydia antigens of the vaccine formulation can be selected from the group described in WO 99/28475.

Preferred bacterial vaccines comprise antigens derived from Streptococcus spp, including S. pneumoniae (PsaA, PspA, streptolysin, choline-binding proteins) and the protein antigen Pneumolysin (Biochem Biophys Acta, 1989, 67, 1007; Rubins et al., Microbial Pathogenesis, 25, 337-342), and mutant detoxified derivatives thereof (WO 90/06951; WO 99/03884). Other preferred bacterial vaccines comprise antigens derived from Haemophilus spp., including H. influenzae type B (for example PRP and conjugates thereof), non typeable H. influenzae, for example OMP26, high molecular weight adhesins, P5, P6, protein D and lipoprotein D, and fimbrin and fimbrin derived peptides (U.S. Pat. No. 5,843,464) or multiple copy variants or fusion proteins thereof.

The antigens that may be used in the present invention may further comprise antigens derived from parasites that cause Malaria. For example, preferred antigens from Plasmodia falciparum include RTS,S and TRAP. RTS is a hybrid protein comprising substantially all the C-terminal portion of the circumsporozoite (CS) protein of P. falciparum linked via four amino acids of the preS2 portion of Hepatitis B surface antigen to the surface (S) antigen of hepatitis B virus. It's full structure is disclosed in the International Patent Application No. PCT/EP92/02591, published under Number WO 93/10152 claiming priority from UK patent application No. 9124390.7. Other plasmodia antigens that are likely candidates to be components of a multistage Malaria vaccine are P. faciparum MSP1, AMA1, MSP3, EBA, GLURP, RAP1, RAP2, Sequestrin, PfEMP1, Pf332, LSA1, LSA3, STARP, SALSA, PfEXP1, Pfs25, Pfs28, PFS27/25, Pfs16, Pfs48/45, Pfs230 and their analogues in Plasmodium spp.

The invention contemplates the use of an anti-tumour antigen and will be useful for the immunotherapeutic treatment of cancers. For example, tumour rejection antigens such as those for prostrate, breast, colorectal, lung, pancreatic, renal or melanoma cancers. Exemplary antigens include MAGE 1, 3 and MAGE 4 or other MAGE antigens such as disclosed in WO99/40188, PRAME, BAGE, Lage (also known as NY Eos 1) SAGE and HAGE (WO 99/53061) or GAGE (Robbins and Kawakami, 1996, Current Opinions in Immunology 8, pps 628-636; Van den Eynde et al., International Journal of Clinical & Laboratory Research (submitted 1997); Correale et al. (1997), Journal of the National Cancer Institute 89, p293. Indeed these antigens are expressed in a wide range of tumour types such as melanoma, lung carcinoma, sarcoma and bladder carcinoma.

MAGE antigens for use in the present invention may be expressed as a fusion protein with an expression enhancer or an Immunological fusion partner. In particular, the Mage protein may be fused to Protein D from Heamophilus influenzae B. In particular, the fusion partner may comprise the first 1/3 of Protein D. Such constructs are disclosed in Wo99/40188. Other examples of fusion proteins that may contain cancer specific epitopes include bcr/abl fusion proteins.

In a preferred embodiment prostate antigens are utilised, such as Prostate specific antigen (PSA), PAP, PSCA (PNAS 95(4) 1735-1740 1998), PSMA or antigen known as Prostase.

Prostase is a prostate-specific serine protease (trypsin-like), 254 amino acid-long, with a conserved serine protease catalytic triad H-D-S and a amino-terminal pre-propeptide sequence, indicating a potential secretory function (P. Nelson, Lu Gan, C. Ferguson, P. Moss, R. Gelinas, L. Hood & K. Wand, “Molecular cloning and characterisation of prostase, an androgen-regulated serine protease with prostate restricted expression, In Proc. Natl. Acad. Sci. USA (1999) 96, 3114-3119). A putative glycosylation site has been described. The predicted structure is very similar to other known serine proteases, showing that the mature polypeptide folds into a single domain. The mature protein is 224 amino acids-long, with one A2 epitope shown to be naturally processed.

Prostase nucleotide sequence and deduced polypeptide sequence and homologs are disclosed in Ferguson, et al. Proc. Natl. Acad. Sci. USA 1999, 96, 3114-3119) and in International Patent Applications No. WO 98/12302 (and also the corresponding granted patent U.S. Pat. No. 5,955,306), WO 98/20117 (and also the corresponding granted patents U.S. Pat. No. 5,840,871 and U.S. Pat. No. 5,786,148) (prostate-specific kallikrein) and WO 00/04149 (P703P).

The present invention provides vectors that encode antigens comprising prostase protein fusions based on prostase protein and fragments and homologues thereof (“derivatives”). Such derivatives are suitable for use in therapeutic vaccine formulations which are suitable for the treatment of a prostate tumours. Typically the fragment will contain at least 20, preferably 50, more preferably 100 contiguous amino acids as disclosed in the above referenced patent and patent applications.

A further preferred prostate antigen is known as P501S, sequence ID no 113 of WO98/37814. Immunogenic fragments and portions encoded by the gene thereof comprising at least 20, preferably 50, more preferably 100 contiguous amino acids as disclosed in the above referenced patent application, are contemplated. A particular fragment is PS108 (WO 98/50567).

Other prostate specific antigens are known from Wo98/37418, and WO/004149. Another is STEAP PNAS 96 14523 14528 7-12 1999.

Other tumour associated antigens useful in the context of the present invention include: Plu-1 J. Biol. Chem. 274 (22) 15633-15645, 1999, HASH-1, HasH-2, Cripto (Salomon et al Bioessays 199, 21 61-70,U.S. Pat. No. 5,654,140) Criptin U.S. Pat. No. 5,981,215. Additionally, antigens particularly relevant for vaccines in the therapy of cancer also comprise tyrosinase and survivin.

The present invention is also useful in combination with breast cancer antigens such as Muc-1, Muc-2, EpCAM, her 2/Neu, mammaglobin (U.S. Pat. No. 5,668,267) or those disclosed in WO/00 52165, WO99/33869, WO99/19479, WO 98/45328. Her 2 neu antigens are disclosed inter alia, in U.S. Pat. No. 5,801,005. Preferably the Her 2 neu comprises the entire extracellular domain (comprising approximately amino acid 1-645) or fragmants thereof and at least an immunogenic portion of or the entire intracellular domain approximately the C terminal 580 amino acids. In particular, the intracellular portion should comprise the phosphorylation domain or fragments thereof. Such constructs are disclosed in WO00/44899.

The her 2 neu as used herein can be derived from rat, mouse or human. The vaccine may also contain antigens associated with tumour-support mechanisms (e.g. angiogenesis, tumour invasion), for example tie 2, VEGF.

Vaccines of the present invention may also be used for the prophylaxis or therapy of chronic disorders in addition to allergy, cancer or infectious diseases. Such chronic disorders are diseases such as asthma, atherosclerosis, and Alzheimers and other auto-immune disorders. Vaccines for use as a contraceptive may also be considered.

Potential human self-antigens or human proteins that modulate the immune response that could include: cytokines, hormones; growth factors or extracellular proteins, more preferably a 4-helical cytokine, most preferably IL13. Cytokines. include, for example, IL1, IL2, IL3, IL4, IL5, IL6, IL7, IL8, IL9, IL10, IL11, IL12, IL13, IL14, IL15, IL16, IL17, IL18, 120, IL21, TNF, TGF, MCSF and OSM. 4-helical cytokines include IL2, IL3, IL4, IL5, IL13, GMCSF and MCSF. Hormones include, for example, luteinising hormone (LH), follicle stimulating hormone (FSH), chorionic gonadotropin (CG), VGF, GHrelin, agouti, agouti related protein and neuropeptide Y.

The vaccines of the present invention are particularly suited for the immunotherapeutic treatment of diseases, such as chronic conditions and cancers, but also for the therapy of persistent infections. Accordingly the vaccines of the present invention are particularly suitable for the immunotherapy of infectious diseases, such as those caused by Human Immunodeficiency Virus (HIV, wherein the antigens are preferably a fusion of RT, nef and gag, optionally further comprising gp120), Hepatitis B and Hepatitis C (wherein the antigens are preferably selected from, or is a combination of, core, NS3, NS4B and NS5B), and Human Papilloma virus (wherein the antigens are preferably E1 and E2, derived from Types 6, 11, 16, 18, and 31, 33, 39, 45, 51, 52, 53, 56, 58, 59, 66 and, other types involved in causing HPV associated disease.

In an embodiment of the invention the antigen is a polynucleotide and is administered/delivered as “naked” DNA, for example as described in Ulmer et al., Science 259:1745-1749, 1993 and reviewed by Cohen, Science 259:1691-1692, 1993. Here the DNA is formulated in a buffered saline solution. The uptake of naked DNA may be increased by coating the DNA onto biodegradable beads or naturally eliminated, which are efficiently transported into the cells or by using other well known transfection facilitating agents. DNA encoding the antigen may be administered in conjunction with a carrier such as, for example, liposomes. Typically such liposomes are cationic, for example imidazolium derivatives (WO95/14380), guanidine derivatives (WO95/14381), phosphatidyl choline derivatives (WO95/35301), piperazine derivatives (WO95/14651) and biguanide derivatives.

Vectors according to the invention which express antigenic peptides may be used as the basis of DNA vaccine compositions and immunotherapeutic compositions. In a similar manner, vectors that encode therapeutic proteins may be used as the basis of therapeutic compositions. Thus, the invention further provides for use of an expression vector according to the invention which is suitable for expression of an antigenic peptide for the manufacture of an immunotherapeutic, vaccine or vaccine composition. The invention further provides a method of vaccinating a mammalian subject which comprises administering thereto an effective amount of such a vaccine or vaccine composition. Most preferably, expression vectors for use in DNA vaccines, vaccine compositions and immunotherapeutics will be plasmid vectors.

DNA vaccines may be administered in the form of “naked DNA”, for example in a liquid formulation administered using a syringe or high pressure jet, or DNA formulated with liposomes or an irritant transfection enhancer, or by particle mediated DNA delivery (PMDD). All of these delivery systems are well known in the art. The vector may be introduced to a mammal for example by means of a viral vector delivery system.

The compositions of the present invention can be delivered by a number of routes such as intramuscularly, subcutaneously, intraperitonally or intravenously.

In a preferred embodiment, the vector is delivered intradermally. In particular, the vector is delivered by means of a gene gun (particularly particle bombardment) administration techniques which involve coating the vector on to a bead (eg gold) which are then administered under high pressure into the epidermis; such as, for example, as described in Haynes et al, J Biotechnology 44: 3742 (1996). There is provided, therefore, gold beads which are suitable for delivery into the epidermis by gene gun delivery which have been coated with the vectors of the present invention.

In one illustrative example, gas-driven particle acceleration can be achieved with devices such as those manufactured by Powderject Pharmaceuticals PLC (Oxford,UK) and Powderject Vaccines Inc. (Madison, Wis.), some examples of which are described in U.S. Pat. Nos. 5,846,796; 6,010,478; 5,865,796; 5,584,807; and EP Patent No. 0500 799. This approach offers a needle-free delivery approach wherein a dry powder formulation of microscopic particles, such as polynucleotide, are accelerated to high speed within a helium gas jet generated by a hand held device, propelling the particles into a target tissue of interest, typically the skin. The particles are preferably gold beads of a 0.4-4.0 μm, more preferably 0.6-2.0 μm diameter and the DNA conjugate coated onto these and then encased in a cartridge or cassette for placing into the “gene gun”.

In a related embodiment, other devices and methods that may be useful for gas-driven needle-less injection of compositions of the present invention include those provided by Bioject, Inc. (Portland, Oreg.), some examples of which are described in U.S. Pat. Nos. 4,790,824; 5,064,413; 5,312,335; 5,383,851; 5,399,163; 5,520,639 and 5,993,412. The present invention provides, therefore, a transdermal powder delivery device for delivering DNA coated beads into the skin of a patient, the delivery device being loaded with beads onto which is coated a vector as described herein.

In one embodiment of the present invention the DNA vaccines comprising the vectors as described herein, are administered in combination with an imiquimod adjuvant. The vectors which comprise the nucleotide sequences encoding antigenic peptides are administered in such amount as will be prophylactically or therapeutically effective. The quantity to be administered, is generally in the range of one picogram to 1 milligram, preferably 1 picogram to 10 micrograms for particle-mediated delivery, and 10 micrograms to 1 milligram for other routes of nucleotide per dose. The exact quantity may vary considerably depending on the species and weight of the mammal being immunised, the route of administration.

It is possible for the immunogen component comprising the nucleotide sequence encoding the antigenic peptide, to be administered on a once off basis or to be administered repeatedly, for example, between 1 and 7 times, preferably between 1 and 4 times, at intervals between about 1 day and about 18 months. Once again, however, this treatment regime will be significantly varied depending upon the size and species of animal concerned, the disease which is being treated/protected against, the amount of nucleotide sequence administered, the route of administration, and other factors which would be apparent to a skilled veterinary or medical practitioner.

It is an embodiment of the invention that the vectors of the invention be utilised with immunostimulatory agents. Preferably the immunostimulatory agent are admisinstered at the same time as the nucleic acid vector of the invention and in preferred embodiments are formulated together. Such iminunostimulatory agents include, but this list is by no means exhaustive and does not preclude other agents: synthetic imidazoquinolines such as imiquimod [Aldara™, S-26308, R-837], (Harrison, et al. ‘Reduction of recurrent HSV disease using imiquimod alone or combined with a glycoprotein vaccine’, Vaccine 19: 1820-1826, (2001)); and resiquimod [S-28463, R-848] (Vasilakos, et al. ‘Adjuvant activites of immune response modifier R-848: Comparison with CpG ODN’, Cellular immunology 204: 64-74 (2000).), Schiff bases of carbonyls and amines that are constitutively expressed on antigen presenting cell and T-cell surfaces, such as tucaresol (Rhodes, J. et al. ‘Therapeutic potentiation of the immune system by costimulatory Schiff-base-forming drugs’, Nature 377: 71-75 (1995)), cytokine, chemoline and co-stimulatory molecules as either protein or peptide, this would include pro-inflammatory cytokines such as GM-CSF, IL1 alpha, IL-1 beta, TGF-alpha and TGF-beta, Th1 inducers such as interferon gamma, IL-2, IL12, IL-15 and IL-18, Th2 inducers such as IL-4, IL-5, IL 6, IL110 and IL-13 and other chemokine and co-stimulatory genes such as MCP-1, MIP-1 alpha, MIP-1 beta, RANTES, TCA-3, CD80, CD86 and CD40L, other immunostimulatory targeting ligands such as CTLAA and L-selectin, apoptosis stimulating proteins and peptides such as Fas, (49), synthetic lipid based adjuvants, such as vaxfectin, (Reyes et al.: ‘Vaxfectin enhances antigen specific antibody titres and maintains Th1 type immune responses to plasmid DNA immunization’, Vaccine 19: 3778-3786) squalene, alpha-tocopherol, polysorbate 80, DOPC and cholesterol, endotoxin, [LPS], Beutler, B., ‘Endotoxin, ‘Toll-like receptor 4, and the afferent limb of innate immunity’, Current Opinion in Microbiology 3: 23-30 (2000)); CpG oligo- and di-nucleotides, Sato, Y. et al., ‘Immunostimulatory DNA sequences necessary for effective intradermal gene immunization’, Science 273 (5273): 352-354 (1996). Hemmi, H. et al., ‘A Toll-like receptor recognizes bacterial DNA’, Nature 408: 740-745, (2000) and other potential ligands that trigger Toll receptors to produce Th1-inducing cytokines, such as synthetic Mycobacterial lipoproteins, Mycobacterial protein p19, peptidoglycan, teichoic acid and lipid A.

Certain preferred adjuvants for eliciting a predominantly Th1-type response include, for example, a Lipid A derivative such as monophosphoryl lipid A, or preferably 3-de-O-acylated monophosphoryl lipid A. MPL® adjuvants are available from Corixa Corporation (Seattle, Wash.; see, for example, U.S. Pat. Nos. 4,436,727; 4,877,611; 4,866,034 and 4,912,094). CpG-containing oligonucleotides (in which the CpG dinucleotide is unmethylated) also induce a predominantly Th1 response. Such oligonucleotides are well known and are described, for example, in WO 96/02555, WO 99/33488 and U.S. Pat. Nos. 6,008,200 and 5,856,462. Immunostimulatory DNA sequences are also described, for example, by Sato et al., Science 273:352, 1996. Another preferred adjuvant comprises a saponin, such as Quil A, or derivatives thereof, including QS21 and QS7 (Aquila Biopharmaceuticals Inc., Framingham, Mass.); Escin; Digitonin; or Gypsophila or Chenopodium quinoa saponins.

One important aspect of the present invention, therefore, is a method of preventing or treating a disease by administering to an individual susceptible or suffering from said disease, a vector according to the present invention in an amount sufficient to raise a prophylactically or therapeutically effective immune response against said disease.

There is also provided the use of the vectors of the present invention in the manufacture of a medicament for the treatment of disease.

The invention further provides host cells transformed or transfected with an expression vector according to the invention. The host cell may be essentially any eukaryotic cell, mammalian cells being most preferred.

The invention still further provides a process for the production of a recombinant polypeptide in a eukaryotic host cell, comprising introducing an expression vector according to the invention into the host cell and culturing the cell under conditions which allow for expression of the polypeptide.

Throughout this specification the terms “comprising”, “comprises” or “comprising of” are to be read inclusively, that is to say that the embodiment includes that stated element but may also include other elements. In an alternate embodiment of the present invention the terms “comprising”, “comprises” or “comprising of” may be substituted with the terms “consisting”, “consists” or “consisting of” which are to be interpreted in the exclusive sense.

The present specification describes many variable parameters, each of which being described as a genus, with various dependent species being described for each. It is intended that this specification discloses all possible combinations of genus and/or species. One vector of the present invention comprises a US3 promoter which consists of the R2 enhancer element, the US3 minimal promoter element which is truncated downstream of the transcription initiation site and the exon 1 sequence from HCMV MIE protein promoter.

The present invention is exemplified by, but not limited to, the following examples.

### Experimental Studies

These investigational studies involved cloning the US3 minimal promoter and R2 region enhancer from the Toledo strain of HCMV into a firefly luciferase reporter protein expression assay system (Promega corp) for comparative expression studies with the HCMV MIE promoter. In addition, comparative expression studies using model antigen OVAcyt and an HIV polyprotein fusion antigen RNG were also undertaken.

Two variants of the US3 enhancer promoter were generated. One comprising the natural US3 DNA sequences designated US3, and one designated US3ex in which the HMCV MIE exon 1 region is fused in place of the US3 untranslated leader sequence (+15 to +81 bp)

### EXAMPLE 1

### Generation of US3 Promoter Fragment from the Toledo Strain of HCMV

The DNA sequence region derived from HCMV strain Toledo and comprising the US3 minimal promoter and R2 enhancer element were cloned by PCR into luciferase reporter vector pGL2 (Promega Corp). Primer pairs for cloning were designed using the US3 DNA sequence from HCMV strain AD 169.

The DNA sequence of HCMV strain AD169 from −346 to +74 (relative to the transcription start site+1) containing the enhancer/promoter (R2 and minimal promoter regions) are shown below. NF-kB domains are in bold. The TATAA box and EcoRV restriction sites are underlined. The CRS element defined in Lashmit et al 1998 is shown in bold.

(SEQ ID NO.1)

CCCGGGTCCCCTCATGCCCTATCGGGATATCGCCGTGTAATGGGGGTGGG
CGACTGACGTGACTCTTGACGTTTATAAACCGCATGGGAAAGTACGGTGT
CGCCACCGTTGACGTGGGCGGCGATGAGAACGTCAGCGGTGGCGAAACCG
CCGTGCGGAAAGTCCCGGTGCCGAAATCACCGTGTGAAAAGTCCCGGTGT
TGAAACCGCCGTGTGGAAAGTCCCGGTTTGGAAATCCCAGTACGGAAAGT
ACCGTAACGCCTCTTTTGGCACGTAGTTGCCTACTACGTAGGGGAAACAA
CGTCACCAAGAAACGCTATATATTCAAAAACACCGTTCAGTCCACACG⁺¹
CTACTTCTCAGCGAAGCACTGCTGCAGCCAGACCGGAGCGGTGAGCGGAG
CCGAGCAGCGGACCTTCGGAGCC

To clone the US3 promoter region from HCMV Toledo PCR primers based on the AD 169 strain were designed and obtained from MWG-biotech AG. A cosmid library generated at GSK and comprising the Toledo. strain HCMV genome was used as source genetic material. The primer sequences and PCR conditions are shown below:

US3 5′ end:

(SEQ ID NO.2)

5′-GGGGTACCCTGCAGCCCGGGTCCCCTCA-3′
US3 3′ end:

(SEQ ID NO.3)

5′-CCCAAGCTT [symbol] GCTCCGAAGGTCCGCT-3′
Kpn I, Pst I, Hind III,

[symbol]

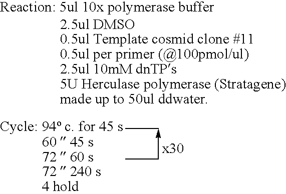

A PCR band of the expected size (˜430 bp) was identified by ethidium staining on an agarose gel. The band was gel purified and restriction digested with Kpn I and Hind III for subsequent ligation into the luciferase reporter vector pGL3 (Promega corp).

### Generation of US3ex Promoter Fragment

The HCMV MIE exon 1 gene sequence was fused immediately after the transcription initiation sequence (ACGCTACTTCT (SEQ ID NO. 4)) of the US3 promoter.

Exon 1 sequence from HGMV MIE CMV promoter

(SEQ ID NO.5)

CGTCAGATCGCCTGGAGACGCCATCCACGCTGTTTTGACCTCCATAGAAG
ACACCGGGACCGATCCAGCCTCCGCGGCCGGGAACGGTGCATTGGAACGC
GGATTCCCCGTGCCAAGAG

The procedure involved four separate PCRS. Oligonucleotides were again obtained from MWG-Biotech AG;

a) PCR for US3 fragment with a fusion sequence at its 3′ end:The PCR conditions & cycle times were as described previously, except that the primers used were US3 5′ and US3/exR;

US3/exR:

(SEQ ID NO.6)

5′-CGTCTCCAGGCGATCTGACGAGAAGTAGCGTGTGGACTG-3′
Exon 1 US3

The PCR produced the correct size fragment which was again purified.
b) 2× PCR for the exon 1 fragment with a fusion sequence at its 5′ end: The PCR conditions & cycle times were as follows;

Oligonucleotides:
US3/exF:

(SEQ ID NO. 7)

5′-CAGTCCACACGCTACTTCTCGTCAGATCGCCTGGAGACG-3′
US3 Exon 1
FVprex F2:

(SEQ ID NO. 8)

5′-TGACCTCCATAGAAGACACCGGGACCGATCCAGCCTCCGCGGCG
G-3′
FVprex F3:

(SEQ ID NO. 9)

5′-GTGCATTGGAACGCGGATTCCCCGTGCCAAGAGGCGGCCGC [symbol]
GGG-3′
FVprex R1:

(SEQ ID NO. 10)

5′-GAATCCGCGTTCCAATGCACCGTTCCCGGCCGCGGAGGCTGGAT
C-3′
FVprex R2:

(SEQ ID NO. 11)

5′-GGTGTCTTCTATGGAGGTCAAAACAGCGTGGATGGCGTCTCCAG
G-3′

All the oligonucleotides were resuspended in water to a concentration of 100 pmol/μl.

An oligonucleotide pool was made by mixing 5 μl of each oligo. This was then used in the following PCR reaction;

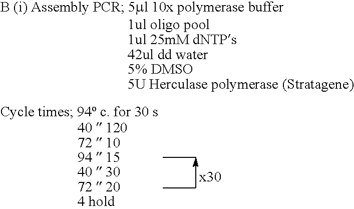

The reaction products were checked on an agarose gel and 100 ul used for the next PCR reaction;

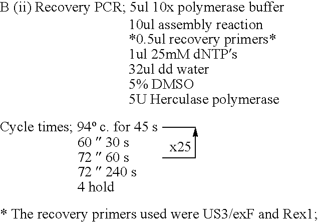

Rex1:

(SEQ ID NO. 12)

5′-CCC [symbol] GCGGCCGCCTCTTGGCACGGGGAATCCGCGTTCCAATG
CAC-3′

Again, the correct sized fragment ˜100 bp for Exon 1 was identified and purified.

C) The final PCR was as follows;

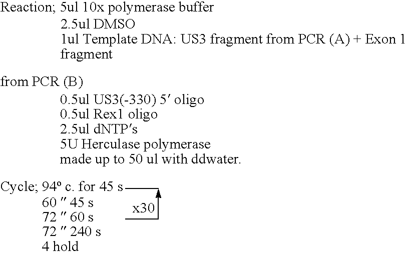

A PCT product band of the expected size (˜500 bp) was identified on an ethidium stained agarose gel. The band was gel purified and restriction enzyme digested with Kpn I and Hind III for subsequent ligation into the luciferase reporter vector pGL3 (Promega corp).

### EXAMPLE 2

### Generation of US3 and US3ex Luciferase Reporter Constructs

Promoter fragments US3 and US3ex were restricted and ligated into vector pGL3 prior to and transformation into bacterial strain JM109. Six bacterial colonies per promoter were selected for insert DNA sequencing. Plasmid DNA was generated form each clone and the insert DNA sequence determined using the following primers;

| | | |
|---|---|---|
| Luc R; | 5′-ATGAGATGTCAGGAACGTCT-3′ | (SEQ ID NO. 13) |
| Luc F; | 5′-TAAGGGATTTTGCCGATTTC-3′ | (SEQ ID NO. 14) |
| RV3; | 5′-CTAGCAAAATAGGGCTGTCCC-3′ | (SEQ ID NO. 15) |

DNA sequence data of the Toledo US3 region from (−350 to +74) reveals several base changes compared to reference strain sequence AD169. Nine of these base changes were consistent across all of the 12 promoter clones studied and are therefore likely to reflect sequence specific differences between HCMV strains Toledo and the reference strain AD169. These nine base changes are shown underlined in US3 promoter clones US3#3 and US3ex#1. No other base changes occur in these clones in comparison to the AD 169 sequence. Clone #3, therefore is the first description of the US3 minimal promoter sequence and the R2 enhancer from the Toledo strain of HCMV. In addition, the TATAA box, and EcoRV restriction site (GATAT) are bold. The CRS element defined in Lashmit 1998 is bold and from this the transcription start is also indicated at (+1).

US3 promoter, clone #3

(SEQ ID NO. 16)

CCCGGGTCCCCTCATGCCCTATCAGGATATCGCCGTGTACTGGGGGTGGG
CGACTGACGTGACTCTTGACGTTTATAAACCGCATGGGAAAGTACGGTGT
CGCCACCGTTGACGTGGGCGGCGATGAAAACGTCAGCGGTGGCGAAACCG
CCGTGCGGAAAGTCCCGGTGGCGAAATCACCGTGCGGAAAGTCCCGGTGT
TGAAACCGCCGTGTGGAAAGTCCCGGTTTGGAAATCCCAGTACGGAAAGT
ACCGTAACGCCTCTTTTGGCACGTAGTTGCCTACTACGTAGGGGAAACAA
CGTCACCAAGAAACGCTATATATCCAAAACCACCGTGCAGTCCACACG⁺¹
CTACTTCTCAGCGAAGCACTGCTGCAGCCAGACCAGAGCGGTGAGCGGAG
CCGAGCAGCGGACCTTCGGAGCC
US3exon 1 promoter, clone #1

(SEQ ID NO. 17)

CCCGGGTCCCCTCATGCCCTATCAGGATATCGCCGTGTACTGGGGGTGGG
CGACTGACGTGACTCTTGACGTTTATAAACCGCATGGGAAAGTACGGTGT
CGCCACCGTTGACGTGGGCGGCGATGAAAACGTCAGCGGTGGCGAAACCG
CCGTGCGGAAAGTCCCGGTGGCGAAATCACCGTGCGGAAAGTCCCGGTGT
TGAAACCGCCGTGTGGAAAGTCCCGGTTTGGAAATCCCAGTACGGAAAGT
ACCGTAACGCCTCTTTTGGCACGTAGTTGCCTACTACGTAGGGGAAACAA
CGTCACCAAGAAACGCTATATATCCAAAACCACCGTGCAGTCCACACGCT
ACTTCTCGTCAGATCGCCTGGAGACGCCATCCACGCTGTTTTGACCTCCA
TAGAAGACATCGGGACCGATCCAGCCTCCGCGGCCGGGAACGGTGCATTG
GAACGCGGATTCCCCGTGCCAAGAG

### EXAMPLE 3

### Analysis of Promoter Activities in Human Cell Line HEK293T

Plasmids were analysed for promoter activity in-vitro using the firefly luciferase assay.

Briefly, transformed human-embryonic kidney 293 cells (BEK293T) were plated out in 96-well black plates (with clear flat bottom wells) at 1×10⁴ cells per well. These were left in a 37° C. incubator overnight. The following day cells were transfected with promoter plasmids@ 250 ng per well using lipofectamine 2000 reagent (Invitrogen) according to manufacturers instructions. Cells were left for 24 hrs before assaying for luciferase activity.

Assay details and reagents can be found in the technical manual TM052 from Promega corp. Plates were read on a Wallac Victor plate reader, and results recorded as relative light units per second. Plasmids were transfected in duplicate. Results are shown in FIG. 1.

Interestingly the 12 promoter clones gave a wide range of luciferase activities, perhaps due to the various mutations within each promoter.

Only four of the 12 clones just have the nine Toledo-specific DNA sequence changes. These are clones US3 #3, #5 and, US3ex #1, #3. Clones US3 #3 and clone US3ex #1 were selected for further studies.

### Kinetics of Promoter Activity

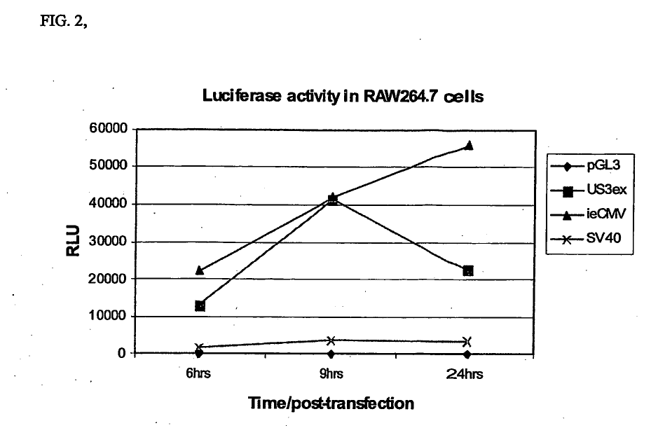

Prior to immunogenicity studies in mice the activity of the US3ex #1 promoter was studied in a murine macrophage cell line. Marine RAW264.7 cells were plated out in 24 well plates and transfected with 0.5 ug of each plasmid vector using Superfect reagent according to the manufacturers instructions (Qiagen). At intervals of 6 hours, 9 hours and 24 hours transfected cells were harvested and luciferase assays undertaken as described above. Average results are shown in FIG. 2.

Expression from the US3ex promoter as determined by luciferase activity is higher than the SV40 promoter and comparable to that of the iCMV promoter at both 6 hours and 9 hours post transfection, At 24 hours the expression of the US3 promoter is lower than iCMV. These kinetics of US3 promoter activity are consistent with the literature.

### The US3 Promoter is Active in Human Dendritic Cells

An important target cell for a therapeutic DNA vaccine is the dendritic cell. Human dentritic cells were isolated from healthy donors and transfected by electroporation using Amaxa Biosystems technology and methods. Four different promoter constructs were studied. Luciferase assays were undertaken 24 hrs after transfection.

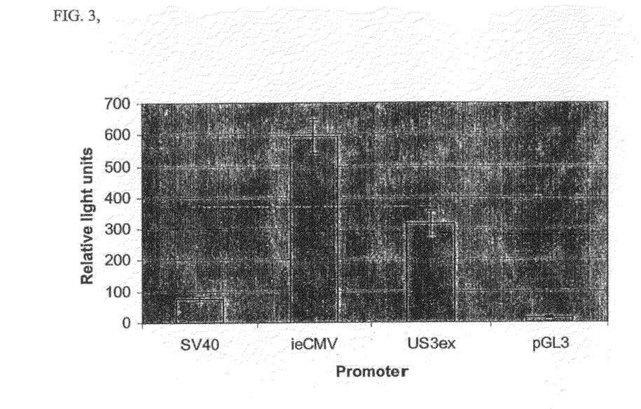

Overall the expression levels in human dendritic cells is lower than in other cell lines such as 293, and may be due to a generally lower level of cellular transfection. However, detectable expression was measured from all three promoters in the human dendritic cells. In this experiment the US3 promoter has intermediate activity between the iCMV and SV40 promoters. For results, see FIG. 3.

### EXAMPLE 4

### US3 Promoter Immunogenicity Studies in Mice Using Model Antigen Ovacyt

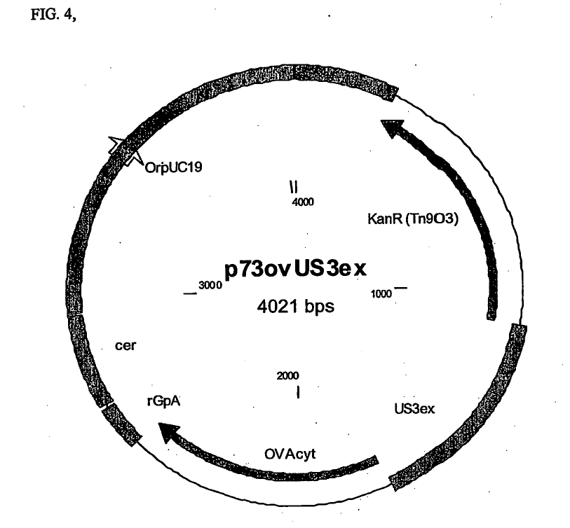

Ovacyt is a model antigen engineered for cytoplasmic rather than nuclear cellular location. The ability of the US3ex promoter to drive gene expression and to evoke an immune response in animals was evaluated using this antigen. The US3ex promoter was cloned behind the Ovacyt gene in vector p7313.ova cyt generating p73OvaUS3ex (see map (FIG. 4). Mice were immunised with plasmid vectors loaded onto gold beads and delivered into the skin using PMID vaccine technology.

Preparation of Cartridges for PMID DNA Immunisation

Preparation of Cartridges for the Accell Gene Transfer Device was as Previously described (Eisenbraun et al DNA and Cell Biology, 1993 Vol 12 No 9 pp 791-797; Pertner et al). Briefly, plasmid DNA was coated onto 2 μm gold particles (DeGussa Corp., South Plainfield, N.J., USA) and loaded into Tefzel tubing, which was subsequently cut into 1.27 cm lengths to serve as cartridges and stored desiccated at 4° C. until use. In a typical vaccination, each cartridge contained 0.5 mg gold coated with plasmid vector to provide a total of 0.5 μg DNA/cartridge.

Immunisation

Plasmid vectors were administered using PMID (0.5 μg/cartridge) into the skin of mice. Plasmid was delivered to the shaved target site of abdominal skin of C57Bl/6 mice (purchased from Charles River United Kingdom Ltd, Margate, UK) from two cartridges using the Accell gene transfer device at 500 lb/in2 (McCabe WO 95/19799).

Immune Assays

Antigen specific T-cell responses were measured using ELISPOT assays. Measurements were taken 7 and 14 days after the priming immunisation. Mice were killed by cervical dislocation and spleens were collected into ice-cold PBS. Splenocytes were teased out into phosphate buffered saline (PBS) followed by lysis of red blood cells (1 minute in buffer consisting of 155 mM NH₄Cl, 10 mM KHCO₃, 0.1 mM EDTA). After two washes in PBS to remove particulate matter the single cell suspension was aliquoted into ELISPOT plates previously coated with capture IFN-γ antibody and stimulated with CD8-restricted peptide. After overnight culture, IFN-γ producing cells were visualised by application of anti-murine IFN-γ-biotin labelled antibody (Pharmingen) followed by streptavidin-conjugated alkaline phosphatase and measured using image analysis.

The amino acid sequence of the peptides used in ELISPOT assays are:

(SEQ ID NO. 18)

| | |
|---|---|
| CD8 restricted Ova peptide | SIINFEKL |

(SEQ ID NO. 19)

| | |
|---|---|
| CD8 restricted GAG peptide | AMQMLKETI |

(SEQ ID NO. 20)

| | |
|---|---|
| CD8 restricted RT peptide | YYDPSKDLI |

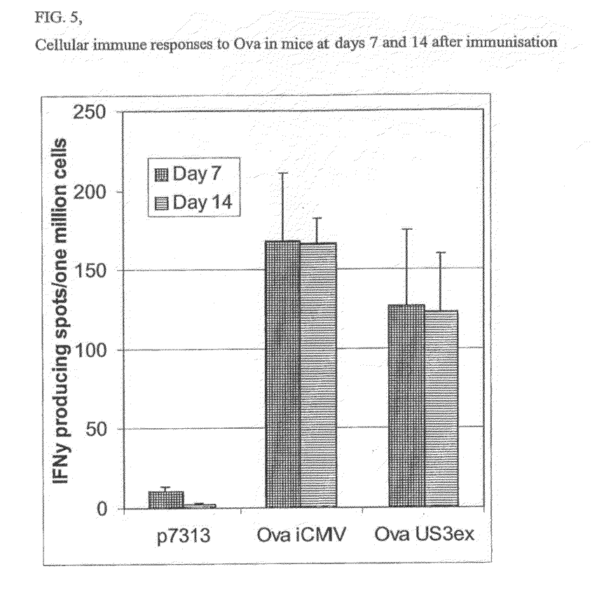

The results of this experiment (FIG. 5) show that the US3ex promoter is active and can lead to the generation of a cellular immune response in mice 7 and 14 days after a single priming immunisation. The level of immune response using the US3ex promoter is comparable to that of the iCMV promoter.

### EXAMPLE 5

### US3 Promoter Immunogenicity Studies in Mice Using HIV Antigens

The utility of the US3 promoter was also evaluated using a fusion protein comprising three HIV antigens, RT, Nef and Gag. These proteins are fused and expressed as a single polyproprotein (RNG).

Vector Construction

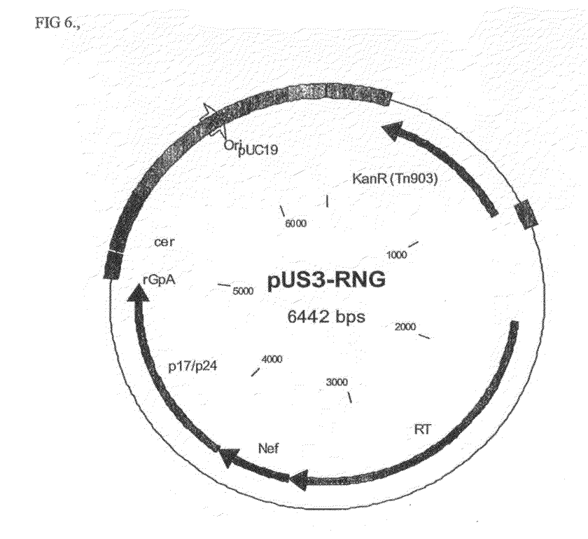

The US3ex promoter was released from the pUS3ex-ova plasmid using restriction enzymes ClaI & NotI to give a fragment of 737 bp. This fragment was used to replace the ClaI-NotI fragment of plasmid pT-RNG. This positioned the RNG polyprotein under control of the US3 promoter so generating plasmid vector pUS3-RNG (see FIG. 6).

The sequence of the RNG insert and methods of its production are described in WO 03/025003 (FIG. 18) the contents of which are incorporated herein by reference.

Note: pT-RNG is a pUC based plasmid designed to express a fusion protein comprising HIV 1.(HXB2) RT (inactive), Nef (truncated), and Gag (p17/24) under the control of an enhanced HCMV MIEE promoter with exon 1 but without intron A. The RT and Gag components have been codon optimised for enhanced mammalian expression.

Immunisation and Immunogenicty Measurements to GAG and RT

Cartridge preparation, immunisation and immune assays were as described above.

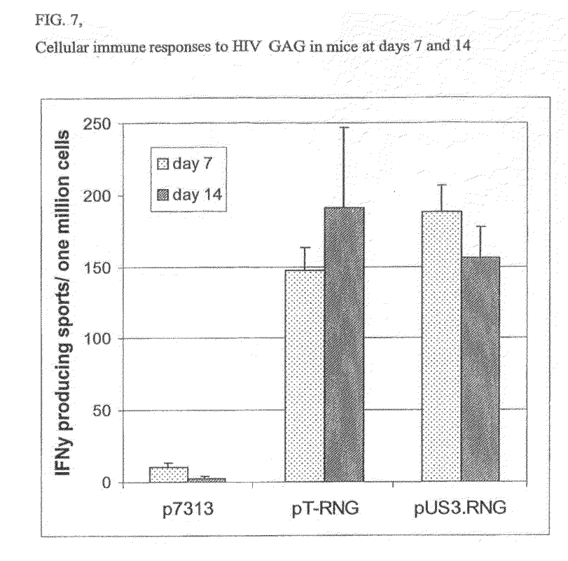

Cellular immune responses to HIV GAG in mice at days 7 and 14 are shown in FIG. 7.

CD8 T-cell responses specific to the Gag peptide are detectable at days 7 and 14 after immunisation. The level of response is comparable to that from the iCMV promoter vector T-RNG.

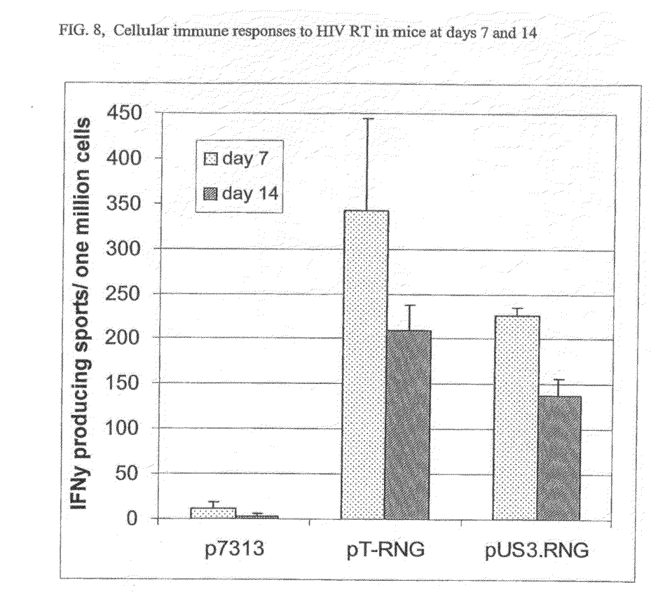

CD8 T-cells specific for HIV RT antigen are also detectable at both days 7 and 14. Cellular immune responses to HIV RT in mice at days 7 and 14 are shown in FIG. 8.

### EXAMPLE 6

### Minipig Promoter Study

### Aims of This Study

Compare immunogenicity of RNG in plasmids (the same plasmids as used in the mouse study described previously) where expression is driven by the US3 promoter (clone #1—with the exon 1 sequence from HCMV MIE gene) with expression driven by HCMV MIE promoter (also including the exon 1 sequence).
Elucidate the kinetics of the immune responses to the three plasmids in the pig.

### Animals

10 minipigs (24 months old, males)

### Groups

Group A CMV promoter-RNG (n=5)
Group B US3ex promoter-RNG (n=5)

### Plasmids for PMID

p7313-RNG with CMV promoter
p7313-RNG with US3ex promoter.

Each cartridge contained 1.0 μg RNG plasmid coated onto 0.5 mg gold beads (2 μm diameter). The gold beads were delivered at 500 psi of helium gas flow.

Schedule

| | | Procedure | | Topical |
|---|---|---|---|---|
| Wk | Day | Blood sample | Immunisation or treatment | Aldara ™ |
| 0 | 0 | ✓ | PMID (Groups A, B and C) | |
| | | | Reactogenicity scores at 1 | |
| | | | and 4 hours post-boost | |
| 0 | 1 | | Reactogenicity scores | ✓ |
| | | | (am + pm) | |
| 8 | 57 | ✓ | PMID (Groups A, B and C) | |
| | | | Reactogenicity scores at 1 | |
| | | | and 4 hours post-boost | |
| 8 | 58 | | Reactogenicity scores | ✓ |
| | | | (am + pm) | |
| 10 | 70 | ✓ | — | |
| 12 | 84 | ✓ | — | |
| 24 | | ✓ | — | |

Immediately prior to immunisations by PMID, immunisation sites were clipped using standard veterinary clippers, cleaned with a clean damp cloth then wiped with a mediwipe swab. Four cartridges per immunisation were delivered at non-overlapping sites on the caudal part of the ventral abdomen. Sites were orientated 1 inch either side of the mid line of the abdomen for primary immunisation and 3 inches either side of the mid line of the ventral abdomen for boost immunisation (i.e. to avoid immunisation over the same area of skin at prime and boost immunisations).

### Aldara

24 hours after each PMID immunisation 20 μl Aldaram (3M) will be applied topically to each site of immunisation. The cream will be rubbed into the skin using a spatula. Aldara™ is a topically applied cream sold for the treatment of genital warts, and contains imiquiimod as the active agent. In this context, imiquimod acts as an adjuvant for the DNA vaccine (for details, see WO 02/24225).

### Blood Sampling

Blood samples (20-40 ml) were collected from each pig into separate 50 ml Falcon tubes containing 1 ml of 1000 U/ml anticoagulant sodium heparin.

### Results

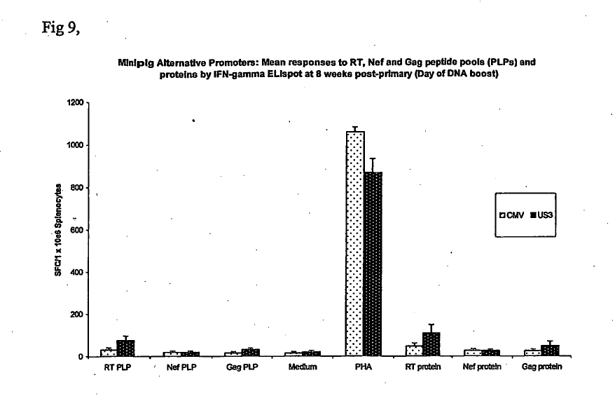

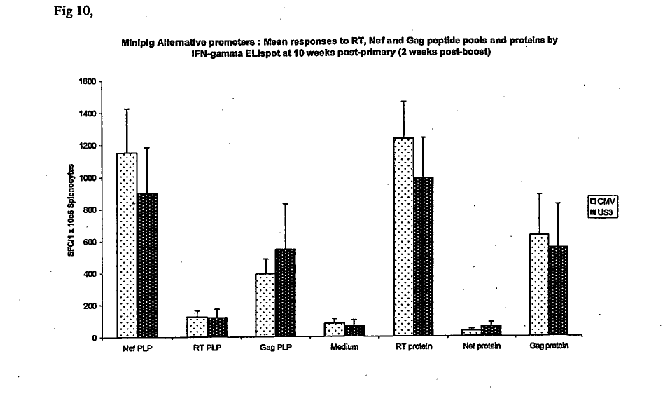

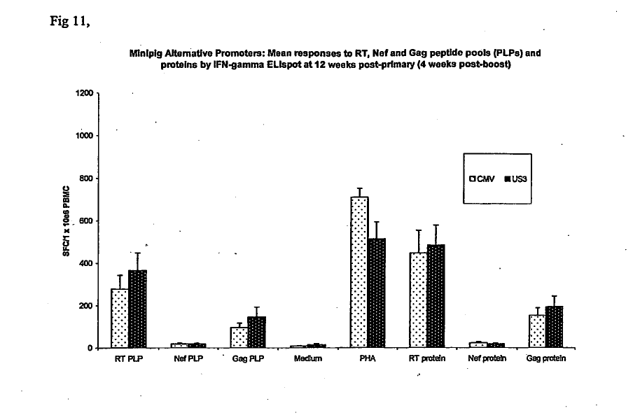

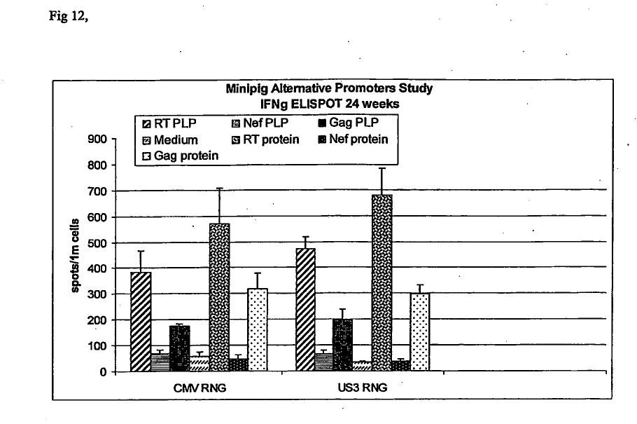

The results of IFN-γ producing cells per million PBMC, as measured in an ELISPOT assay against protein or peptide pools are shown in FIGS. 9 to 12. The results show that the DNA vaccine comprising the US3 promoter generated immune responses in the pigs which were at least equivalent to those induced by the DNA vaccines comprising the HCMV MIE promoter.

1. A polynucleotide vector comprising a promoter element of the Human Cytomegalovirus (HCMV) US3 gene, the promoter being operably linked to a region encoding a heterologous polypeptide which is foreign with respect to the HCMV US3 protein.

2. A polynucleotide vector as claimed in claim 1, comprising the minimal promoter element of the Human Cytomegalovirus (HCMV) US3 gene and a transcription regulatory element, the minimal promoter being operably linked to a region-encoding a heterologous protein which is foreign with respect to HCMV US3.

3. A polynucleotide vector as claimed in claim 2 wherein said transcription regulatory element is an enhancer element.

4. A polynucleotide vector as claimed in claim 3, wherein the enhancer element is the R2 enhancer element from the HCMV US3 gene.

5. A polynucleotide vector as claimed in claim 4 wherein the R2 enhancer element is positioned immediately upstream of the minimal HCMV US3 promoter.

6. A polynucleotide vector as claimed in any one of claims 1 to 5, further comprising the HCMV MIE exon 1 gene sequence fused after the transcription initiation sequence of the US3 promoter.

7. A polynucleotide vector as claimed in claim 1 wherein the silencing effect of the R1 element within the US3 promoter has been reduced or abrogated.

8. An polynucleotide vector as claimed in claim 7 where the sequence of the US3 R1 element has been removed.

9. A polynucleotide vector comprising a promoter having the R2 enhancer element of the HCMV US3 gene promoter, and a minimal promoter element from a non-HCMV US3 gene promoter.

10. A polynucleotide vector as claimed in claim 9, wherein the minimal promoter element from a non-HCMV US3 gene promoter is the HCMV MIE gene minimal promoter element.

11. A polynucleotide vector according to any one of claims 1 to 10 which is plasmid vector.

12. A polynucleotide vector according to any one of claims 1 to 11 which is an expression vector for use in expression of a polypeptide in a eukaryotic host cell or organism.

13. A polynucleotide vector according to claim 12 wherein the polypeptide is an antigenic polypeptide.

14. A polynucleotide expression vector according to claim 13 for use as a vaccine or immunotherapeutic or as a component of a vaccine composition or immunotherapeutic composition.

15. A polynucleotide expression vector according to claim 12 for use in the in vitro expression of a therapeutic protein.

16. An immunogenic composition comprising a polynucleotide expression vector according to any one of claims 1 to 14 and a pharmaceutically acceptable adjuvant diluent, excipient or carrier.

17. An immunogenic composition according to claim 16 which carrier comprises a bead onto which the vector is coated.

18. Use of a polynucleotide expression vector according to any one of claims 1 to 14 in the manufacture of a vaccine, immunotherapeutic, vaccine composition or immunotherapeutic composition.

19. A method of vaccinating a human subject which comprises administering to said subject an effective amount of a vaccine or vaccine composition comprising an expression vector according to claim 14, or composition according to claim 16 or 17.

20. A host cell transformed or transfected with a polynucleotide expression vector according to claim 15.

21. A process for the production of a recombinant polypeptide in a eukaryotic host cell, comprising introducing an expression vector as claimed in claim 15 into the host cell under conditions which allow for expression of the polypeptide.

22. A transdermal powder delivery device for delivering DNA coated beads into the skin of a patient, the delivery device being loaded with beads onto which is coated a vector as claimed in any one of claims 1 to 14.

23. A polynucleotide vector as claimed in any one of claims 1 to 14 for use in gene therapy.


SEQUENCE LISTING (24 sequences)

SEQ ID NO: 1 - DNA, 421 bp, human cytomegalovirus
cccgggtcccctcatgccctatcgggatatcgccgtgtaatgggggtgggcgactgacgtgactcttgacgtttataaaccgcatgggaaagtacggtgtcgccaccgttgacgtgggcggcgatgagaacgtcagcggtggcgaaaccgccgtgcggaaagtcccggtgccgaaatcaccgtgtgaaaagtcccggtgttgaaaccgccgtgtggaaagtcccggtttggaaatcccagtacggaaagtaccgtaacgcctcttttggcacgtagttgcctactacgtaggggaaacaacgtcaccaagaaacgctatatattcaaaaacaccgttcagtccacacgctacttctcagcgaagcactgctgcagccagaccggagcggtgagcggagccgagcagcggaccttcggagcc

SEQ ID NO: 2 - DNA, 27 bp, human cytomegalovirus
ggggtaccctgcagcccgggtccccta

SEQ ID NO: 3 - DNA, 34 bp, human cytomegalovirus
cccaagcttgcggccgcggctccgaaggtccgct

SEQ ID NO: 4 - DNA, 11 bp, human cytomegalovirus
acgctacttct

SEQ ID NO: 5 - DNA, 119 bp, human cytomegalovirus
cgtcagatcgcctggagacgccatccacgctgttttgacctccatagaagacaccgggaccgatccagcctccgcggccgggaacggtgcattggaacgcggattccccgtgccaagag

SEQ ID NO: 6 - DNA, 39 bp, Artificial Sequence
  primer HCMV
cgtctccaggcgatctgacgagaagtagcgtgtggactg

SEQ ID NO: 7 - DNA, 39 bp, Artificial Sequence
  primer HCMV
cagtccacacgctacttctcgtcagatcgcctggagacg

SEQ ID NO: 8 - DNA, 45 bp, Artificial Sequence
  primer HCMV
tgacctccatagaagacaccgggaccgatccagcctccgcggccg

SEQ ID NO: 9 - DNA, 50 bp, Artificial Sequence
  primer HCMV
gtgcattggaacgcggattccccgtgccaagaggcggccgcaagcttggg

SEQ ID NO: 10 - DNA, 45 bp, Artificial Sequence
  primer HCMV
gaatccgcgttccaatgcaccgttcccggccgcggaggctggatc

SEQ ID NO: 11 - DNA, 45 bp, Artificial Sequence
  primer HCMV
ggtgtcttctatggaggtcaaaacagcgtggatggcgtctccagg

SEQ ID NO: 12 - DNA, 50 bp, Human Cytomegalovirus
  luciferase
cccaagcttgcggccgcctcttggcacggggaatccgcgttccaatgcac

SEQ ID NO: 13 - DNA, 20 bp, Artificial Sequence
  luciferase
atgagatgtcaggaacgtct

SEQ ID NO: 14 - DNA, 20 bp, Artificial Sequence
  luciferase
taagggattttgccgatttc

SEQ ID NO: 15 - DNA, 21 bp, Artificial Sequence
  primer HCMV
ctagcaaaatagggctgtccc

SEQ ID NO: 16 - DNA, 421 bp, Human Cytomegalovirus
cccgggtcccctcatgccctatcaggatatcgccgtgtactgggggtgggcgactgacgtgactcttgacgtttataaaccgcatgggaaagtacggtgtcgccaccgttgacgtgggcggcgatgaaaacgtcagcggtggcgaaaccgccgtgcggaaagtcccggtggcgaaatcaccgtgcggaaagtcccggtgttgaaaccgccgtgtggaaagtcccggtttggaaatcccagtacggaaagtaccgtaacgcctcttttggcacgtagttgcctactacgtaggggaaacaacgtcaccaagaaacgctatatatccaaaaccaccgtgcagtccacacgctacttctcagcgaagcactgctgcagccagaccagagcggtgagcggagccgagcagcggaccttcggagcc

SEQ ID NO: 17 - DNA, 475 bp, Human Cytomegalovirus
cccgggtcccctcatgccctatcaggatatcgccgtgtactgggggtgggcgactgacgtgactcttgacgtttataaaccgcatgggaaagtacggtgtcgccaccgttgacgtgggcggcgatgaaaacgtcagcggtggcgaaaccgccgtgcggaaagtcccggtggcgaaatcaccgtgcggaaagtcccggtgttgaaaccgccgtgtggaaagtcccggtttggaaatcccagtacggaaagtaccgtaacgcctcttttggcacgtagttgcctactacgtaggggaaacaacgtcaccaagaaacgctatatatccaaaaccaccgtgcagtccacacgctacttctcgtcagatcgcctggagacgccatccacgctgttttgacctccatagaagacatcgggaccgatccagcctccgcggccgggaacggtgcattggaacgcggattccccgtgccaagag

SEQ ID NO: 18 - protein, 8 aa, Ovalbumin
SIINFEKL

SEQ ID NO: 19 - protein, 9 aa, Human Immunodeficiency Virus
AMQMLKETI

SEQ ID NO: 20 - protein, 9 aa, Human Immunodeficiency Virus
YYDPSKDLI

SEQ ID NO: 21 - DNA, 10 bp, Human Cytomegalovirus
tgtcgcgaca

SEQ ID NO: 22 - DNA, 15 bp, Human Cytomegalovirus
cgtgcagtccacacg

SEQ ID NO: 23 - DNA, 7 bp, Human Cytomegalovirus
ctacttc

SEQ ID NO: 24 - DNA, 11 bp, Human Cytomegalovirus
acgctacttct